(19) United States
(12) Patent Application Publication
(10) Pub. No.: US 2003/0023217 A1
(43) Pub. Date: Jan. 30, 2003

(54) Package for absorbent articles

(75) Inventors: James D. McManus, Appleton, WI (US); Ann M. Nichols, Appleton, WI (US); Paul M. Simons, Appleton, WI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Kimberly-Clark Worldwide, Inc.

(21) Appl. No.: 09/966,065

(22) Filed: Sep. 28, 2001

Related U.S. Application Data
(63) Continuation of application No. 09/917,540, filed on Jul. 27, 2001, now abandoned.

(51) Int. Cl. A61F 13/15; A61F 13/20; A61L 15/00; A61B 17/06; A61B 19/02

(52) U.S. Cl. 604/385.01; 206/438

(57) ABSTRACT

A package including absorbent articles and recloseable packaging. The packaging includes a flexible pocket having a hollow interior for said absorbent articles, an opening extending into the hollow interior for permitting an article to be withdrawn from the hollow interior, and a flap attached to the pocket for covering the opening. The flap may be moved between an open position to permit an article to be withdrawn through the opening and a closed position to retain the articles in the pocket. The packaging also includes a closure positioned on the pocket and/or the flap for releasably holding the flap in the closed position. The closure has an overall length extending between opposite lateral ends thereof which is less than a corresponding width of the pocket.

### CROSS-REFERENCE TO RELATED APPLICATION

This is a continuation of U.S. patent application Ser. No. 09/917,540, filed Jul. 27, 2001, entitled, “PACKAGE FOR ABSORBENT ARTICLES”, which is hereby incorporated by reference in its entirety.

### BACKGROUND OF THE INVENTION

The present invention relates generally to a package, and more particularly to a package for absorbent articles.

Absorbent articles such as panty liners, feminine napkins and tampons are frequently carried about in purses, backpacks and briefcases until needed. Typically, the articles are put in these containers loose. Unfortunately, these containers do not always provide a hygienic environment for the articles, and thus the articles can become dirty and/or damaged. Further, the articles can become scattered about in the containers so they are difficult to find when needed.

In the past, specifically designed pouches have been distributed for holding several absorbent articles at a time. These pouches reduce contact between the articles and potentially non-hygienic environments, and make the articles easier to find when needed. The pouches are made from durable materials such as heavy vinyl so they can be reused, but reuse necessitates the pouches be refilled from time to time. Further, the pouches frequently become non-hygienic after extended use, requiring them to be cleaned or discarded and replaced.

To avoid these problems, some persons use clear plastic sandwich bags to hold the articles. These bags usually maintain a hygienic environment for the articles and make the articles easier to find when needed. Although the bags must be filled from time to time, they are readily disposable and replaceable thereby reducing some of the concerns and inconveniences caused by extended use. However, because the bags are transparent, they permit the contents of the bags to be viewed. Some users desire more discreet packaging. Thus, there is a need for a discreet, disposable and economical recloseable package for holding several articles at a time.

One type of packaging which meets the previously mentioned need is described in co-assigned U.S. patent application Ser. No. 09/713,496, entitled “Package”, filed Nov. 15, 2000, which is hereby incorporated by reference in its entirety. As shown in FIG. 1, this packaging (generally designated by the reference number 10) includes a pocket 12 for holding the articles and an opening (not shown) for withdrawing articles from the pocket when needed. Further, the package 10 includes a rectangular flap 14 for selectively covering the opening. The flap 14 has an adhesive closure 16 (FIG. 3) for holding the flap against the pocket 12 when covering the opening. It has been noted that if the flap 14 is not aligned with the pocket 12 when closed as illustrated in FIGS. 2 and 3, the adhesive closure 16 extends past the pocket where it can become stuck to other objects and/or collect non-hygienic debris. Thus, there is a need for packaging 10 having a closure 16 which permits misalignment between the flap 14 and the pocket 12 without exposing the closure.

### SUMMARY OF THE INVENTION

Briefly, apparatus of this invention is a package comprising a plurality of absorbent articles and recloseable packaging. The packaging includes a flexible pocket having a hollow interior sized and shaped for receiving the plurality of absorbent articles and an opening extending into the hollow interior of the pocket sized and shaped for permitting at least one of the plurality of articles to be withdrawn from the hollow interior of the pocket through the opening. The packaging also includes a flap attached to the pocket adapted for covering the opening to retain the plurality of absorbent articles in the hollow interior of the pocket. The flap is selectably moveable between an open position in which the opening is generally unobstructed by the flap to permit the at least one article to be withdrawn through the opening and a closed position in which the flap covers the opening and an exterior area of the pocket to retain the plurality of articles in the pocket and to prevent the at least one article from passing through the opening. Further, the packaging includes a closure positioned on at least one of the pocket and the flap for releasably holding the flap in the closed position. The closure has an overall length extending between opposite lateral ends thereof which is less than a corresponding width of the pocket.

In another aspect, the present invention includes a package comprising a plurality of absorbent articles and recloseable packaging including a flexible pocket, an opening, a flap, and a closure. The flap may be rotated when in the closed position about an axis extending normal to a surface of the flap by as much as about twenty degrees without exposing the closure.

In still another aspect, the present invention includes a package comprising a plurality of absorbent articles and recloseable packaging including a flexible pocket, an opening, a flap, and a closure. The flap may be rotated when in the closed position about an axis extending normal to a surface of the flap by as much as about twenty degrees without the closure extending past a side of the pocket.

Other features of the present invention will be in part apparent and in part pointed out hereinafter.

### BRIEF DESCRIPTION OF THE DRAWINGS

FIG. 1 is a front elevation of a package for holding panty liners showing a flap of the package in a closed position;

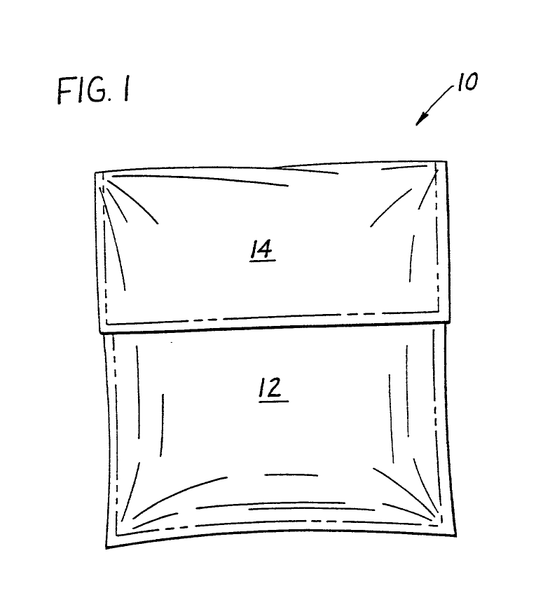

FIG. 2 is a front elevation of the package showing the flap misaligned with a pocket of the package in the closed position;

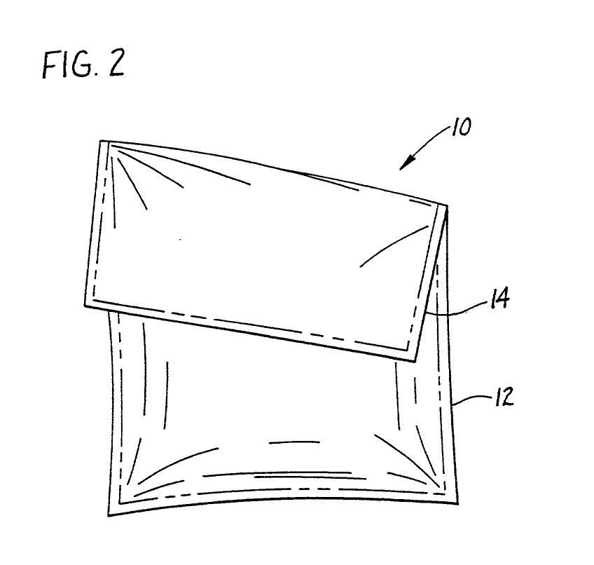

FIG. 3 is a rear elevation of the package showing the flap misaligned with the pocket in the closed position;

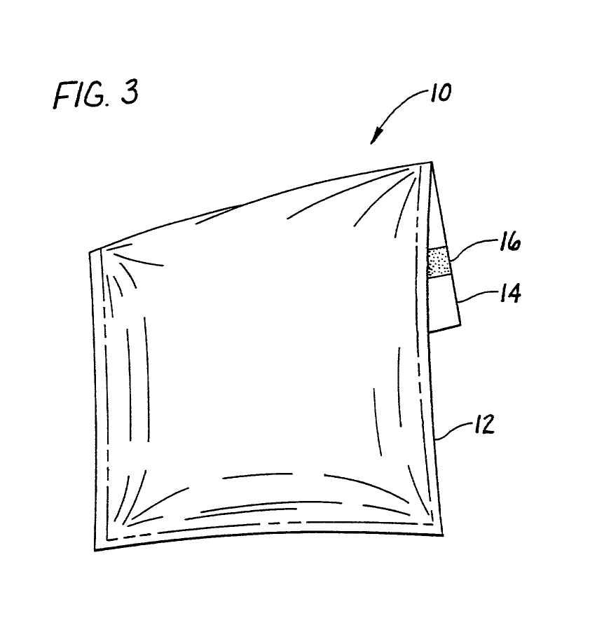

FIG. 4 is a front elevation of a prior art panty liner surrounded by a removable wrapper in partial section;

FIG. 5 is a front elevation of a package of the present invention showing a flap of the package in a closed position;

FIG. 6 is a front elevation of the package showing the flap in an open position;

FIG. 7 is a top plan of the package in partial section; and

FIG. 8 is a front elevation of the package showing the flap misaligned with the pocket in the closed position.

Corresponding reference characters indicate corresponding parts throughout the several views of the drawings.

### DETAILED DESCRIPTION OF THE PREFERRED EMBODIMENT

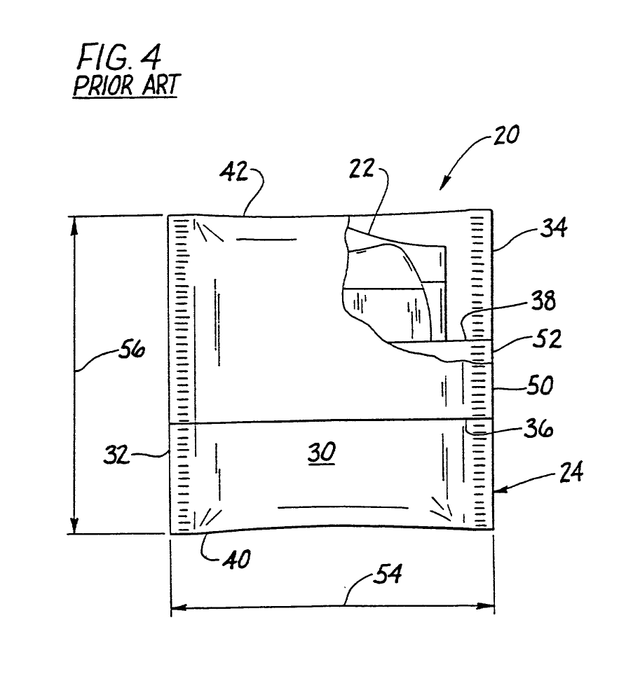

Referring now to the drawings and in particular to FIG. 4, a conventional individually wrapped absorbent article is designated in its entirety by the reference numeral 20. Although the absorbent article 20 shown in FIG. 4 is a panty liner 22 surrounded by a wrapper, generally designated by 24, those skilled in the art will appreciate that the present invention may be applied to other absorbent articles 20 such as feminine napkins, tampons, interlabial pads, other feminine care products, adult care products, child care products and infant care products. Further, those skilled in the art will appreciate that although the illustrated articles 20 are individually wrapped, the articles may be unwrapped without departing from the scope of the present invention.

The panty liner 22 shown in FIG. 4 is folded twice in a conventional manner to present a generally flat and generally rectangular article. Although the folded panty liner 22 may have other sizes without departing from the scope of the present invention, in one embodiment the folded liner has a width of about 50 millimeters, a length of about 70 millimeters and a thickness of about 5 millimeters. It is also envisioned that the panty liner 22 may be unfolded and/or non-rectangular without departing from the scope of the present invention.

Although the wrapper 24 may be made in other ways without departing from the scope of the present invention, in one embodiment the wrapper includes a rectangular sheet 30 having opposite side edges 32, 34 and opposite end edges 36, 38. A first fold 40 in the sheet 30 extending between the side edges 32, 34 forms a bottom of the wrapper 24, and a second fold 42 in the sheet extending between the side edges generally parallel to and above the first fold forms a top of the wrapper. A margin 50 of the sheet 30 adjacent the end edge 36 overlaps a margin 52 of the sheet adjacent the end edge 38. It is envisioned that it may be desirable to join the corresponding side edges 32, 34 to themselves. Although the side edges 32, 34 of the rectangular sheet 30 may be joined in other ways (such as with adhesives or by heat sealing) without departing from the scope of the present invention, in one embodiment the side edge margins are joined by conventional mechanical fastening means as shown.

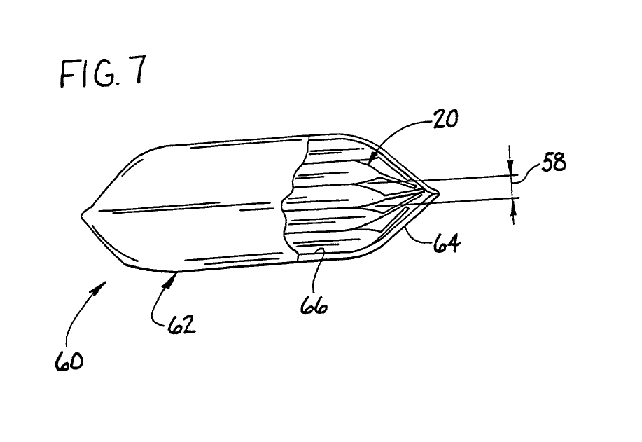

Although the wrapped article 20 may have other sizes without departing from the scope of the present invention, in one embodiment the article has a width 54 of about 75 millimeters, a length 56 of about 75 millimeters and a thickness 58 (FIG. 7) of about 5 millimeters. Further, although the dimensions of the article 20 may vary from article to article without departing from the scope of the present invention, in one embodiment the dimensions are generally uniform. In addition, the dimensions may vary within a given article or they may be invariant without departing from the scope of the present invention. Although the wrapper 24 may be made of other materials without departing from the scope of the present invention, in one embodiment the wrapper is made from low density polyethylene sheet material having a thickness of about 38 microns. It is further envisioned that the wrapper 24 may have an adhesive or other closure (not shown) without departing from the scope of the present invention.

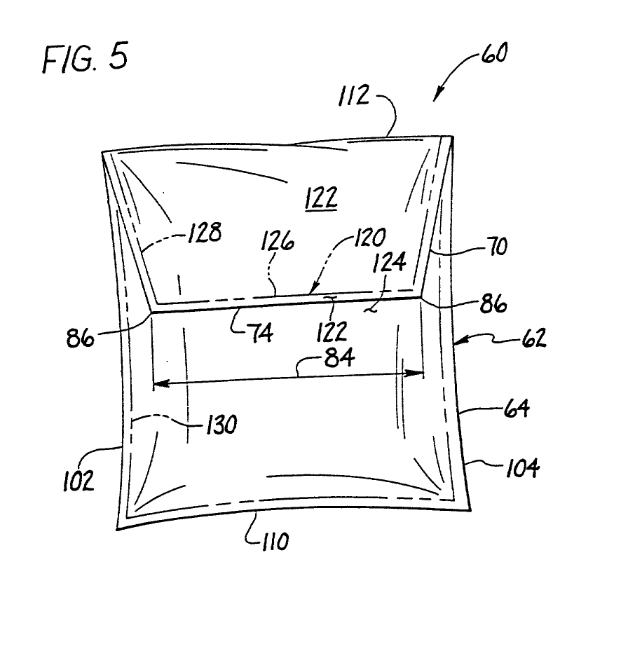

As illustrated in FIGS. 5-7, a utility package of the present invention is designated in its entirety by the reference number 60. The package 60 generally comprises a plurality of absorbent articles 20 (FIG. 4) and recloseable packaging, generally designated by 62. As previously mentioned, it is envisioned that the articles 20 may be folded or unfolded and individually wrapped or unwrapped without departing from the scope of the present invention. Although the articles 20 may be arranged in other ways without departing from the scope of the present invention, in one embodiment the articles are arranged in face-to-face relation in a stack as illustrated in FIG. 7. The package 60 includes a number of articles 20 which is selected for the convenience of an end user. For example, the package 60 may include less than about eight absorbent articles 20 to provide a supply of articles for about one week or less. It is envisioned that it may be desirable that the package 60 include about five absorbent articles 20 to provide a supply of articles for one conventional work week (i.e., five days).

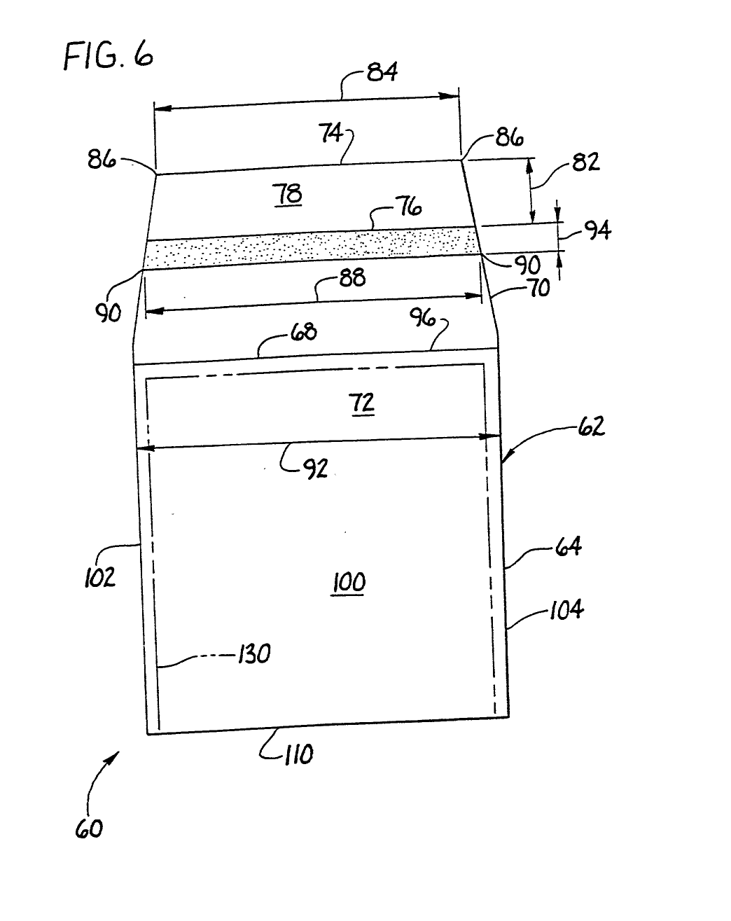

The packaging 62 includes a flexible pocket 64 having a hollow interior 66 sized and shaped for receiving the preselected number of absorbent articles 20 as shown in FIG. 7. An opening 68 (FIG. 6) extends into the hollow interior 66 of the pocket 64. The opening 68 is sized and shaped for permitting at least one of the plurality of articles 20 to be withdrawn from the hollow interior 66 of the pocket 64. Further, the packaging 62 includes a flap 70 attached to the pocket 64 adapted for covering the opening 68 to retain the plurality of articles 20 in the hollow interior 66 of the pocket 64. The flap 70 is selectably moveable between an open position as illustrated in FIG. 6 in which the opening 68 is generally unobstructed by the flap to permit at least one article 20 to be withdrawn through the opening and a closed position as shown in FIG. 5 in which the flap covers the opening and an exterior area 72 (FIG. 6) of the pocket 64 to retain the articles in the pocket and to prevent the articles from passing through the opening. In one embodiment, the flap 70 is trapezoidal and has a substantially straight distal edge 74. However, those skilled in the art will appreciate that the flap 70 may have other shapes such as semi-circular or triangular without departing from the scope of the present invention.

As illustrated in FIG. 6, a closure 76 is positioned on an inner face 78 of the flap 70 for releasably holding the flap in the closed position. It is envisioned that the closure 76 may be positioned on the exterior area 72 of the pocket 64 instead of or in addition to being positioned on the flap 70. Preferably, the closure 76 permits the flap 70 to be positioned in a different location on the pocket 64 during successive closings when successive articles 20 are removed from the interior 66 of the pocket to permit the packaging 62 to conform to the remaining articles in the interior of the pocket. Although other closures 76 may be used without departing from the scope of the present invention, in one embodiment the closure is an adhesive material such as a conventional resealable hot melt adhesive or a resealable two sided tape for releasably holding the flap 70 in the closed position.

Although the closure 76 may be positioned at other locations without departing from the scope of the present invention, in one embodiment the closure is spaced from the distal edge 74 of the flap 70 to permit a free portion of the flap to be grasped and separated from the pocket 64 to grip the flap for releasing the closure. It is envisioned that it may be desirable that the closure 76 be spaced from the distal edge 74 of the flap 70 by a distance 82 of at least about 4 millimeters. It is further envisioned that it may be desirable that the distance 82 be about 16 millimeters.

Although the closure 76 may have other shapes without departing from the scope of the present invention, in one embodiment the closure is an elongate strip of adhesive material extending substantially parallel to the distal edge 74 of the flap 70. In one embodiment, the strip of adhesive material is substantially continuous and uninterrupted along its entire length, but it is envisioned that other embodiments may have adhesive material which is discontinuous and/or interrupted along its length without departing from the scope of the present invention. In addition, it is envisioned that it may be desirable that the strip extend over a distance greater than or equal to a distal width 84 of the flap 70 so the corners 86 of the flap are held in place near the pocket 64. Further, it is envisioned that it may be desirable that the strip extend over an entire width of the flap 70 corresponding to the strip. As shown in FIG. 6, the closure 76 preferably has an overall length 88 extending between opposite lateral ends 90 of the closure which is less than a corresponding width 92 of the pocket 64. Although the closure 76 may have other overall lengths 88 without departing from the scope of the present invention, in one embodiment the overall length of the closure is between about 80 percent and about 95 percent of the width 92 of the pocket 64. More precisely, the overall length 88 of the closure 76 of the one embodiment is about ninety percent of the width 92 of the pocket 64. Still more precisely, the overall length 88 of the one embodiment is about 91 percent of the width 92 of the pocket 64. Although the closure 76 may have other overall lengths 88 without departing from the scope of the present invention, in one embodiment the overall length of the closure is between about five millimeters and about ten millimeters less than the width 92 of the pocket 64. More precisely, the overall length 88 of the closure 76 of the one embodiment is about 7.5 millimeters less than the width 92 of the pocket 64. Although the elongate strip may have other widths without departing from the scope of the present invention, in one embodiment the strip has a width 94 of less than about 20 millimeters and more than about 2 millimeters. It may be desirable that the width 94 of the strip be about 7 millimeters.

Although the packaging 62 may be made in other ways without departing from the scope of the present invention, in one embodiment illustrated in FIG. 6 the packaging comprises a sheet 100 having opposite side edges 102, 104 and opposite end edges 74, 106. A first fold 110 extending between the side edges 102, 104 forms a bottom of the packaging 62, and a second fold 112 (FIG. 5) extending between the side edges generally parallel to and above the first fold forms a top of the packaging. The second fold 112 forms a hinge separating the flap 70 from the pocket 64. Although the second fold 112 may be positioned in other locations without departing from the scope of the present invention, in one embodiment the second fold is positioned adjacent the opening 68 and the end edge 106. Portions of the side edges 102, 104 are joined to form opposite sides of the pocket 64. Although the side edges 102, 104 may be joined in other ways (such as with adhesives or by mechanical fastening) without departing from the scope of the present invention, in one embodiment they are joined by conventional heat sealing. Although the packaging 62 may be made from other materials without departing from the scope of the present invention, in one embodiment the packaging is made from a heat sealable polymer sheet material such as a material containing about 80 percent polyethylene and about 20 percent other polyolefins having a thickness of between about 0.001 inches and about 0.002 inches available from Shanghai Zihua Enterprise Company, Limited of Shanghai, China. It is further envisioned that the packaging 62 may be made from coated paper, woven material, non-woven material, polyethylene, polypropylene, co-polymers, extruded polymer, thermo-formed materials, and/or cardboard without departing from the scope of the present invention. Although in one embodiment the pocket 64 is substantially free of gussets, in an alternate embodiment the sides of the pocket may include conventional gussets (not shown) to provide the packaging 62 with a substantially flat bottom. Although other sequences are envisioned as being within the scope of the present invention, in one embodiment the sheet 100 is printed, the first fold 110 is made and the side edges 102, 104 are heat sealed to form the pocket 64. Once the pocket 64 is formed, the closure 76 is applied to the inner face 78 of the flap 70 and the packaging 62 filled with the plurality of absorbent articles 20. Once the packaging 62 is filled, the second fold 112 is made and the closure 76 is sealed to complete the package 60.

As illustrated in FIG. 5, it may be desirable that the flap 70 and/or the pocket 64 include a visual indicator, generally designated by 120, for distinguishing the edge 74 of the flap from the pocket. The indicator 120 improves visual identification of the edge 74 of the flap 70 when grasping the flap to move it toward its open position. The visual indicator 120 comprises visually contrasting surface treatments on the flap 70 and the pocket 64. Although other visually contrasting surface treatments may be used without departing from the scope of the present invention, in one embodiment the visually contrasting surface treatments include a first color on at least a portion of an exterior surface or outer face 122 of the flap 70 and a second color on at least a portion of an exterior surface 124 of the pocket 64. Further, it may be desirable that one of the colors be a raw material color of the packaging material and the other color be printed on the packaging 62. For example, if the raw material color of the packaging 62 is white, a lower edge margin 126 and opposing side margins 128 of the flap 70 may be substantially free of printing, and a central portion of the flap may include printing of a contrasting color (e.g., blue). The margins 126, 128 are delineated by phantom lines in the drawings. In addition to omitting printing from the edge margins 126, 128 of the flap 70, it may be desirable that the side margins 130 of the pocket 64 (delineated by phantom lines) be substantially free of printing, to prevent printing discoloration when the sides are heat sealed.

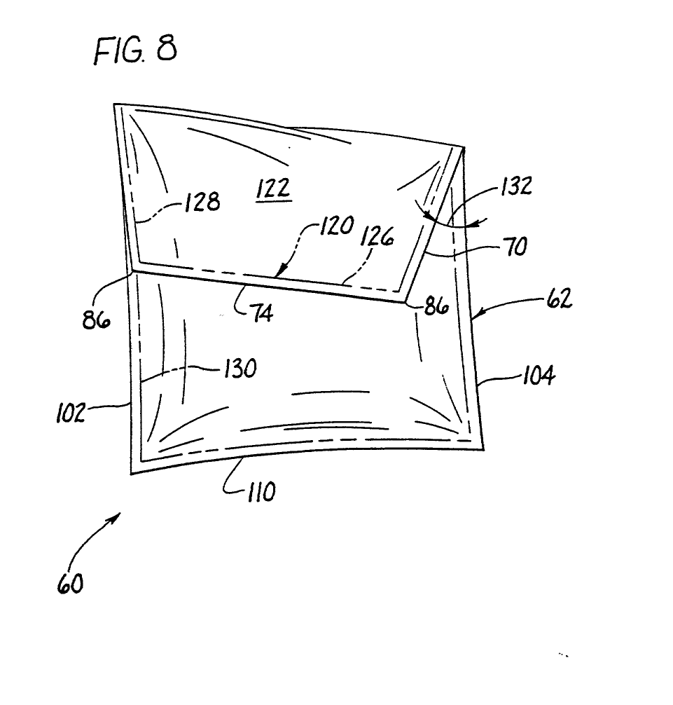

As illustrated in FIG. 8, one advantage of the closure 76 having an overall length 88 which is less than a corresponding width 92 of the pocket 64 is that the flap 70 may be rotated when in the closed position about an axis extending normal to a surface of the flap through an angle 132 without exposing the closure and without the closure extending past a side 102, 104 of the pocket. Although the angle 132 through which the flap 70 is rotatable without exposing the closure or with the closure extending past the sides 102, 104 of the pocket 64 may vary without departing from the scope of the present invention, in one embodiment the flap may be rotated by as much as about twenty degrees without exposing the closure or without the closure extending past a side of the pocket.

In view of the above, it will be seen that the several objects of the invention are achieved and other advantageous results attained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

What is claimed is:

1. A package comprising a plurality of absorbent articles and recloseable packaging including:
a flexible pocket having a hollow interior sized and shaped for receiving said plurality of absorbent articles;
an opening extending into the hollow interior of the pocket sized and shaped for permitting at least one of said plurality of articles to be withdrawn from the hollow interior of the pocket through the opening;
a flap attached to the pocket adapted for covering the opening to retain said plurality of absorbent articles in the hollow interior of the pocket, said flap being selectably moveable between an open position in which the opening is generally unobstructed by said flap to permit said at least one article to be withdrawn through the opening and a closed position in which the flap covers the opening and an exterior area of the pocket to retain said plurality of articles in the pocket and to prevent said at least one article from passing through the opening; and
a closure positioned on at least one of said pocket and said flap for releasably holding said flap in the closed position, said closure having an overall length extending between opposite lateral ends thereof which is less than a corresponding width of the pocket.

2. A package as set forth in claim 1 wherein the overall length of the closure is between about 80 percent and about 95 percent of the width of the pocket.

3. A package as set forth in claim 2 wherein the overall length of the closure is about ninety percent of the width of the pocket.

4. A package as set forth in claim 1 wherein the overall length of the closure is between about five millimeters and about ten millimeters less than the width of the pocket.

5. A package as set forth in claim 4 wherein the overall length of the closure is about 7.5 millimeters less than the width of the pocket.

6. A package as set forth in claim 1 wherein said closure permits the flap to be positioned in a different location on the pocket during successive closings when successive articles are removed from the interior of the pocket to permit said packaging to conform to the remaining articles in the interior of the pocket.

7. A package as set forth in claim 1 wherein the flap has an inner face and an outer face, and the closure is positioned on said inner face of the flap for releasably holding said flap in the closed position.

8. A package as set forth in claim 7 wherein the closure is an adhesive material applied to the inner face of the flap.

9. A package as set forth in claim 1 wherein the closure is spaced from a distal edge of the flap to permit a portion of the flap to be grasped and separated from the pocket without releasing the closure.

10. A package as set forth in claim 1 wherein said closure includes an elongate strip of adhesive material extending substantially parallel to a distal edge of the flap.

11. A package as set forth in claim 10 wherein the strip of adhesive material is substantially continuous and uninterrupted along its entire length.

12. A package as set forth in claim 1 wherein the closure extends substantially an entire width of the flap.

13. A package as set forth in claim 1 wherein the flap may be rotated when in the closed position about an axis extending normal to a surface of the flap by as much as about twenty degrees without exposing the closure.

14. A package as set forth in claim 1 wherein the flap may be rotated when in the closed position about an axis extending normal to a surface of the flap by as much as about twenty degrees without said closure extending past a side of the pocket.

15. A package comprising a plurality of absorbent articles and recloseable packaging including:
a flexible pocket having a hollow interior sized and shaped for receiving said plurality of absorbent articles;
an opening extending into the hollow interior of the pocket sized and shaped for permitting at least one of said plurality of articles to be withdrawn from the hollow interior of the pocket through the opening;
a flap attached to the pocket adapted for covering the opening to retain said plurality of absorbent articles in the hollow interior of the pocket, said flap being selectably moveable between an open position in which the opening is generally unobstructed by said flap to permit said at least one article to be withdrawn through the opening and a closed position in which the flap covers the opening and an exterior area of the pocket to retain said plurality of articles in the pocket and to prevent said at least one article from passing through the opening; and
a closure positioned on at least one of said pocket and said flap for releasably holding said flap in the closed position, wherein the flap may be rotated when in the closed position about an axis extending normal to a surface of the flap by as much as about twenty degrees without exposing the closure.

16. A package as set forth in claim 15 wherein said closure permits the flap to be positioned in a different location on the pocket during successive closings when successive articles are removed from the interior of the pocket to permit said packaging to conform to the remaining articles in the interior of the pocket.

17. A package as set forth in claim 15 wherein the flap has an inner face and an outer face, and the closure is positioned on said inner face of the flap for releasably holding said flap in the closed position.

18. A package as set forth in claim 17 wherein the closure is an adhesive material applied to the inner face of the flap.

19. A package as set forth in claim 15 wherein the closure is spaced from a distal edge of the flap to permit a portion of the flap to be grasped and separated from the pocket without releasing the closure.

20. A package as set forth in claim 15 wherein said closure includes an elongate strip of adhesive material extending substantially parallel to a distal edge of the flap.

21. A package as set forth in claim 20 wherein the strip of adhesive material is substantially continuous and uninterrupted along its entire length.

22. A package as set forth in claim 15 wherein the closure extends substantially an entire width of the flap.

23. A package as set forth in claim 15 wherein the flap may be rotated when in the closed position about an axis extending normal to a surface of the flap by as much as about twenty degrees without said closure extending past a side of the pocket.

24. A package comprising a plurality of absorbent articles, and recloseable packaging including:
a flexible pocket having a hollow interior sized and shaped for receiving said plurality of absorbent articles;
an opening extending into the hollow interior of the pocket sized and shaped for permitting at least one of said plurality of articles to be withdrawn from the hollow interior of the pocket;
a flap attached to the pocket adapted for covering the opening to retain said plurality of absorbent articles in the hollow interior of the pocket, said flap being selectably moveable between an open position in which the opening is generally unobstructed by said flap to permit said at least one article to be withdrawn through the opening and a closed position in which the flap covers the opening and an exterior area of the pocket to retain said plurality of articles in the pocket and to prevent said at least one article from passing through the opening; and
a closure positioned on the flap for releasably holding the flap in the closed position, wherein the flap may be rotated when in the closed position about an axis extending normal to a surface of the flap by as much as about twenty degrees without said closure extending past a side of the pocket.

25. A package as set forth in claim 24 wherein said closure permits the flap to be positioned in a different location on the pocket during successive closings when successive articles are removed from the interior of the pocket to permit said packaging to conform to the remaining articles in the interior of the pocket.

26. A package as set forth in claim 24 wherein the flap has an inner face and an outer face, and the closure is positioned on said inner face of the flap for releasably holding said flap in the closed position.

27. A package as set forth in claim 26 wherein the closure is an adhesive material applied to the inner face of the flap.

28. A package as set forth in claim 24 wherein the closure is spaced from a distal edge of the flap to permit a portion of the flap to be grasped and separated from the pocket without releasing the closure.

29. A package as set forth in claim 24 wherein said closure includes an elongate strip of adhesive material extending substantially parallel to a distal edge of the flap.

30. A package as set forth in claim 29 wherein the strip of adhesive material is substantially continuous and uninterrupted along its entire length.

31. A package as set forth in claim 24 wherein the closure extends substantially an entire width of the flap.
